SEQUENCE LISTING (6 sequences)

SEQ ID NO: 1 - DNA, 492 bp, MAMMALIAN
  misc_feature (1)..(492): PROBE SELECTED FOR THE GAPDH GENE
ctggtgtcttcaccaccatggagaaggccggggcccacttgaagggtggagccaaacgggtcatcatctccgccccttctgccgatgcccccatgtttgtgatgggtgtgaaccacgagaaatatgacaactcactcaagattgtcagcaatgcatcctgcaccaccaactgcttagcccccctggccaaggtcatccatgacaactttggcattgtggaagggctcatgaccacagtccatgccatcactgccacccagaagactgtggatggcccctctggaaagctgtggcgtgatggccgtggggctgcccagaacatcatccctgcatccactggtgctgccaaggctgtgggcaaggtcatcccagagctgaacgggaagctcactggcatggccttccgtgttcctacccccaatgtgtccgtcgtggatctgacgtgccgcctggagaaacctgccaagtatgatgacatcaagaaggtggtga

SEQ ID NO: 2 - DNA, 237 bp, MAMMALIAN
  misc_feature (1)..(237): PROBE SELECTED FOR THE GAPDH GENE
ttcaccaccatggagaaggccggggcccacttgaagggtggagccaaacgggtcatcatctccgccccttctgccgatgcccccatgtttgtgatgggtgtgaaccacgagaaatatgacaactcactcaagattgtcagcaatgcatcctgcaccaccaactgcttagcccccctggccaaggtcatccatgacaactttggcattgtggaagggctcatgaccacagtccatgcc

SEQ ID NO: 3 - DNA, 249 bp, MAMMALIAN
  misc_feature (1)..(249): PROBE SELECTED FOR THE HPRT GENE
gctggtgaaaaggacctctcgaagtgttggatacaggccagactttgttggatttgaaattccagacaagtttgttgttggatatgcccttgactataatgagtacttcaggaatttgaatcacgtttgtgtcattagtgaaactggaaaagccaaatacaaagcctaagatgagcgcaagttgaatctgcaaatacgaggagtcctgttgatgttgccagtaaaattagcaggtgttctagtcctgtg

SEQ ID NO: 4 - DNA, 491 bp, MAMMALIAN
  misc_feature (1)..(491): PROBE SELECTED FOR THE HPRT GENE
aggagatgggaggccatcacattgtggccctctgtgtgctcaaggggggctataagttctttgctgacctgctggattacattaaagcactgaatagaaatagtgatagatccattcctatgactgtagattttatcagactgaagagctactgtaatgatcagtcaacgggggacataaaagttattggtggagatgatctctcaactttaactggaaagaatgtcttgattgttgaagatataattgacactggtaaaacaatgcaaactttgctttccctggttaagcagtacagccccaaaatggttaaggttgcaagcttgctggtgaaaaggacctctcgaagtgttggatacaggccagactttgttggatttgaaattccagacaagtttgttgttggatatgcccttgactataatgagtacttcaggaatttgaatcacgtttgtgtcattagtgaaactggaaaagccaaatacaaagcc

SEQ ID NO: 5 - DNA, 20 bp, ARTIFICIAL SEQUENCE
  PRIMER
ctggtgtcttcaccaccatg

SEQ ID NO: 6 - DNA, 22 bp, ARTIFICIAL SEQUENCE
  PRIMER
tcaccaccttcttgatgtcatc

(19) United States
(12) Patent Application Publication
(10) Pub. No.: US 2004/0106121 A1
(43) Pub. Date: Jun. 3, 2004

(54) Static micro-array of biological or chemical probes immobilised on a support by magnetic attraction

(75) Inventors: Nicolas Ugolin, Paris (FR); Gerard Marguerie De Rotrou, Vitry (FR); Thierry Kortulewski, Chilly-Mazarin (FR); Olivier Alibert, Saint-Germain Le Corbeil (FR); Diana Le Roux, Meudon (FR)

Correspondence Address: BIRCH STEWART KOLASCH & BIRCH, PO BOX 747, FALLS CHURCH, VA, 22040-0747, US

(73) Assignee: COMMISSARIAT A L'ENERGIE ATOMIQUE, Paris (FR)

(21) Appl. No.: 10/444,778

(22) Filed: May 23, 2003

Related U.S. Application Data
(63) Continuation of application No. PCT/FR01/03780, filed on Nov. 29, 2001.

(30) Foreign Application Priority Data
Nov. 29, 2000 (FR) 0015398

(51) Int. Cl. C12Q 1/68; C12M 1/34

(52) U.S. Cl. 435/6; 435/287.2

(57) ABSTRACT

The invention relates to an organized array of biological or chemical probes bonded to a support by magnetic coupling using a fixing vector. The support may include a permanent magnet or present electrically-induced magnetization. The fixing vectors may be constituted by magnetic beads functionalized to be capable of bonding specifically to one particular type of biological probe.

The present invention relates to an array of biological or chemical ligands (probes) fixed by magnetic attraction on a support that is not chemically functionalized.

The probes may be natural or synthetic substances presenting biological or chemical activity or affinity for biological or chemical molecules, e.g. peptides, proteins, oligonucleotides, RNA, single- or double-strand DNA, polysaccharides, and phospholipids, and combinations of chemical substances. Such arrays, which are of very low cost, are applicable in numerous fields, for example as diagnostic tools, or as tools for screening collections of molecules or of biological samples, or of molecules for therapeutic or diagnostic purposes. The invention lies in said ligands being fixed stably on a magnetic surface in order to organize an array of biological or chemical probes. This method can be adapted to producing micro-arrays of various densities, or macro-arrays.

Numerous methods have been proposed over the last few years for making miniature arrays of biological probes fixed in well-defined positions.

There are two technologies for making such matrices. The first technology consists in directly synthesizing short oligonucleotides or peptides on a surface that has previously been functionalized and activated to make grafting possible.

The second technology consists in fixing previously synthesized and characterized ligands on a functionalized surface, with said probes being capable of being deposited by mechanical or electrochemical methods.

Several patents and publications describe the application of photolitography to performing photochemical addressing, in particular in order to obtain a matrix of oligonucleotides or of peptides by photochemical addressing. That method consists in using chemically functionalized surfaces protected by photo-activatable groups. Selective illumination through masks then makes it possible to remove the protection from different coupling sites and to perform in situ synthesis of the oligonucleotide or the peptide.

That method of photochemical addressing presents several drawbacks. Firstly, the efficiency of the reactions to remove protection is not 100%. Consequently, in situ syntheses accumulate errors that can lead, for example, to ligands being truncated or to oligonucleotide terminations being wrongly paired. This requires probes to be highly redundant and numerous checks to be performed, and also requires considerable computer power to reconstitute a standard signal. Furthermore, the quality of interaction or hybridization reactions with the short-length ligands fixed in the solid phase is difficult to predict. Finally, using sets of masks complicates the method and makes it expensive and relatively inflexible, thereby constituting a major drawback. Genome and functional databases are evolving all the time, since they are continuously integrating new results being obtained by very many laboratories. This leads firstly to said databases being enriched, and secondly to certain erroneous sequences that are present in the databases being corrected. In the fields of research and clinical studies, it is therefore useful to be able to adapt the array used to the biological matter under investigation as a function of changing knowledge. The above-described methods for manufacturing arrays which rely on using sets of masks do not have the flexibility needed for rapid modification at low cost of the sequences of certain probes, nor for easy addition of even a single additional probe.

The second type of technology used for producing arrays of probes would appear a priori to be better adapted to integrating new data since it consists in synthesizing and characterizing the probes individually prior to fixing them on a support.

A first method of grafting probes is described in Lehrach et al. (Hybridization fingerprinting in genome mapping and sequencing, Genome Analysis, 1990, Cold Spring Harbor Press, pp. 39-81). That method uses needles for building up arrays of 9216 hybridization units. The limit of that method lies in the great variability of the deposits and the small number of arrays that can be produced at a time.

Other technologies using mechanical or electrochemical addressing have been described. In those methods, the ligands are prepared, purified, and then deposited on an activated surface. For example, oligonucleotides of various lengths and preamplified double-strand probes can be deposited in order to make DNA matrix arrays. The deposition surface may be made of glass or of optionally porous silicon, of polymer material, or of any other chemically functionalized surface.

Thus, several documents describe using the micro-pipetting or needle-transfer technique for depositing nucleic probes on glass surfaces functionalized by poly-l-lysine or by an activated polyacrylamide gel with certain amide groups being substituted by hydrazide groups. Those methods present several major drawbacks. Firstly, chemically functionalizing the surface is difficult to control, but without such functionalizing, robust covalent fixing of the ligand is not possible. In addition, the density of ligands that are deposited and fixed is not controllable. Finally, in some cases, chemically grafting the probes can spoil their reactivity relative to their targets, for example in the event of a receptor being fixed to a functionalized support by means of an amine group close to the recognition site.

Electrochemical addressing techniques have also been described (patent application WO A-94/22889). That case starts with a support having a plurality of electrodes, and the electrodes are used for fixing biological ligands electrochemically, making use of ligands that have previously been functionalized by means of an electrolyte such as the pyrrole ring which has the property of polymerizing under the effect of an electric current. That method presents the advantage of controlled addressing of ligands over a reaction zone. Nevertheless, the addressing makes it necessary to use a conductive surface, functionalized reagents, and a process that is difficult to industrialize and provide quality control for matrices produced in great quantities.

All of the methods described above enable micro-arrays of biological probes to be made, but they are limited both concerning their suitability for diversification and because they are often complex to implement on an industrial scale.

In addition, most of the documents cited above describe using those methods to obtain arrays of nucleic probes that are restricted to oligonucleotides or to double-strand DNA. Nevertheless, for reasons of sensitivity, it is more advantageous to use single-strand DNA probes of length greater than that of synthetic oligonucleotides, which are difficult to make more than about ten oligonucleotides long. For example, single-strand DNA probes having 100 to 500 bases would make it possible to obtain excellent hybridization specificity. Furthermore, the advent of functional genomics has led to a high level of demand for arrays of polypeptide, protein, and even cellular probes.

It can thus be seen that there exists a large need for methods that are less expensive, better adapted to numerous modifications, capable of using all types of probe (nucleic, protein, cellular, chemical, . . . ), and which can be made on demand in laboratories without requiring special equipment.

An object of the present invention is specifically to provide micro-arrays of extremely varied probes which may be nucleic probes (oligonucleotides, double-strand DNA or single-strand DNA, RNA, . . . ), proteins (membrane receptors, monoclonal antibodies, peptides, recombinant proteins, or domains thereof, . . . ), or even viruses, cells, or organically synthesized molecules coming from combinational libraries. Such arrays of biological or chemical probes are easy to make, using a method which relies on fixing probes on a support by magnetic interaction. The matrices created in this way can be used in all of the fields in which micro- or macro-arrays are used, and more particularly in diagnosis, pharmacogenomics, toxicogenomics, studying the structure and the expression of genomes, and in general in any type of application involving molecular interactions. They can thus be used in particular as tools for diagnosis or for high speed screening. Manufacturing these arrays does not require any prior chemical functionalization of the support and can be performed in a manner that is simple and inexpensive. The quality of the chemical substrate on which fixing takes place is no longer a limiting element.

One of the advantages of the invention is also that of increasing the detection sensitivity of such arrays. A significant example lies in the possibility of fixing single-strand DNA, thereby avoiding double-strand competition during hybridization of nucleic targets. The ability to fix a plurality of fragments or domains of recombinant proteins easily after prior testing for specific activities constitutes another significant example. The absence of chemical treatment for grafting probes makes it possible to perform serial optical processing on slides so as to make them inert to fluorescence and to diffusion interference.

The detailed description of the invention relies on various notions which are defined below:

Throughout the present text, the term “biological probes” designates:

any biological molecule such as peptides, proteins, and glycoproteins (antigens, antibodies, receptors, ligands, or fragments thereof), nucleic acids (single- or double-strand DNA, oligonucleotides, RNA), carbohydrates, lipoproteins, lipids;

any type of cell (bacteria, protozoa, yeast, fungi, eukaryotic cells, . . . );

any type of virus; or

fragments of cells or viruses, etc.

“Chemical probes” designate any type of chemical molecule, for example coming from a combinational library.

In this application, a “fixing vector” is an element that is capable firstly of bonding covalently or by strong interactions to a biological and/or chemical probe, and that is capable secondly of being fixed to a support by magnetic attraction. In one particular case, a fixing vector is a magnetic bead of the type known to the person skilled in the art and commonly used in biology in order to separate cells or to purify molecules, e.g. as sold by Miltenyi or Dynabeads (e.g. Dynal M-280 streptavidin). The fixing vector is not necessarily spherical. In all cases, the “diameter of the fixing vector” designates the diameter of the sphere within which the particle can be circumscribed.

An “organized array” can be defined as a set of spots such that the content of each spot is known as a function of its coordinates. By extension, the term “array” is used herein to designate a set of spots fixed on a support, in which case it becomes synonymous with “chip”.

A “magnetic chip” is a chip of the invention, i.e. an array of biological or chemical ligands or probes fixed on a support by magnetic attraction by means of a fixing vector.

In the present invention, a “spot” of an array constitutes a hybridization or reaction unit carrying a given probe, and presenting given “density”.

“Spot density” is defined herein as being the number of probes per spot (e.g. the number of molecules if the probe is molecular).

“Array density” designates the number of spots per unit area.

A “paramagnetic” material is characterized by low magnetic susceptibility with rapid loss of magnetization once no longer in a magnetic field.

“Ferromagnetic” materials have high magnetic susceptibility and are capable of conserving magnetic properties in the absence of a magnetic field (permanent magnetism).

So-called “superparamagnetic” materials are characterized by high magnetic susceptibility (i.e. they become strongly magnetic when they are placed in a magnetic field), but like paramagnetic materials, they lose their magnetization quickly in the absence of the magnetic field. Superparamagnetism can be obtained in ferromagnetic materials when the size of the crystal is smaller than a critical value. Superparamagnetic beads present the dual advantages of being capable of being subjected to strong attraction by a magnet, and of not clumping together in the absence of a magnetic field.

The support may be constituted by a magnetized slide, thus creating a magnetic field of uniform intensity over its surface. Alternatively, the support may present “structured magnetization” which is defined by a magnetic field of intensity that is not uniform over the surface of the support, presenting maxima in well-defined positions. This applies for example with a silica slide having micromagnets embedded therein (three-dimensional structuring), or to a simple magnetized slide covered by a mask that is opaque to magnetic field and that is pierced by regularly spaced-apart windows (two-dimensional structuring).

The present invention consists in fixing biological or chemical probes in well-defined positions organized in an array on a magnetic support, the probes being connected by a covalent bond or by strong interactions to a particle capable of being attracted by a magnetic force. Said particles constitute the “fixing vectors”. The probes are held at precise coordinates on the support by interaction between the fixing vectors and the magnetic support.

The support must thus be capable of supplying or being subjected to magnetic attraction. It may be constituted by a simple magnetized slide, however it may also act as a structured support having three-dimensional magnetic zones, e.g. a silica slide having cavities filled with ferromagnetic particles. The magnetization of the support may also be of electrical origin. An example of a support presenting structured electromagnetization is a microcircuit having a set of current-carrying conductor loops.

The fixing vectors for coupling probes to the support are elements capable of being subjected to and possibly also of providing magnetic attraction. These fixing vectors may be paramagnetic, i.e. attracted by a magnet but not presenting magnetization when taken out of the magnetic field. However the coupling between the probes and the support by means of the fixing vectors of paramagnetic material is not very strong, and it is therefore preferable to envisage using superparamagnetic or ferromagnetic materials. By way of example these can be magnetic beads having a diameter of less than 5 micrometers (μm) as are commonly used in biology and are inexpensive, or else nanoparticles such as nanoferrins.

The probe(s) can be fixed to the fixing vector either by covalent bonds, or by non-covalent bonds of the affinity type, for example, such as the bonds involved between streptavidin and biotin, the bonds between antigens and antibodies, or receptor/ligand interactions. Numerous types of magnetic beads covered with molecules capable of bonding specifically with a biological target are already on sale. For example there are beads covered with streptavidin or in avidin that are capable of interacting with biotin, or beads carrying particular antibodies capable of bonding with proteins, or with cells by interacting with a membrane receptor. Beads covered with poly-T or poly-U oligonucleotides can also be used as a fixing vector for nucleic acid probes including a poly-A residue (e.g. cDNA having a poly-A “tail” or messenger RNA). These examples are not limiting in any way and the person skilled in the art knows how to select other fixing vectors that are adapted to the application intended for the arrays described herein.

Each array spot, or hybridization unit, represents an identified area on the support of a few tens of square micrometers (μm²) to 1 square millimeter (mm²), where identical probes are grafted that are fixed by interacting with their fixing vector. The diameter of the array spots depends on the type of fixing vector used and is selected as a function of the intended application. Thus, in a DNA chip for diagnosis or high throughput screening, the array spots are advantageously of small size, e.g. having a diameter of abut 50 μm. In contrast, a larger diameter, e.g. 200 μm, is preferable in other types of application, for example when the probes are cells. In preferred manner, all of the spots of a given array have the same area. The invention thus applies to a two-dimensional or three-dimensional array of spots regularly disposed on the support, each spot being constituted by a different type of biological or chemical probe connected by covalent bonds or strong interactions with its fixing vector, itself fixed on the support by magnetic interaction.

The originality of such an array lies in the method used for fixing each pair constituted by a probe and its fixing vector at precise coordinates on the support in order to form a micro-array. The probes are fixed in the array not by means of a chemical reaction, but by relying on a physical force: magnetic force.

Probes coupled to the support by magnetic interaction via a fixing vector presents numerous advantages. Firstly, this type of coupling avoids any prior chemical treatment of the support for the purpose of fixing the probes, and it does not require storage to be performed under special conditions. If the surface of the support is subjected to chemical functionalization, then the random nature of the surface treatment and the damage that occurs during storage mean that it is not possible to guarantee that the chemical substrate is reproducible and stable. By way of example, mention can be made of the dehydration or the rehydration of a substrate of lysine or of polyacrylamide. With arrays of the kind described in the present invention, the magnetic supports can be used immediately and they do not require any special packaging.

In addition, the absence of any need to subject the support to chemical treatment in order to fix the probes makes it possible to cover the surface of the support in a substance that is optically neutral and permeable to magnetic fields, in order to optimize detection.

The molecular probes can be constituted by a molecule of RNA or of single- or double-strand DNA, proteins, or peptides, and more generally by any type of biological or chemical molecule that is to be fixed in highly stable manner at precise positions on a support in order to constitute a micro-array. In the context of studying nucleic acids, the size and the quality of the single-strand fragments of DNA or RNA that are used is not limiting since they are synthesized and purified away from the chip under optimum conditions. In a particular embodiment of an array of the invention, as illustrated in Example 4, the probes are single-strand DNA molecules of large size, thereby increasing detection sensitivity compared with oligonucleotide probes or with double-strand DNA probes.

Fixing probes on the support by means of a fixing vector that is coupled to the support by magnetic interaction also makes it very easy to make arrays of (optionally genetically modified) cells in order to screen molecules. For example, cells that have been genetically modified to express stress genes can be fixed to screen stress agents or antistress molecules in large series.

Durable fixing of the molecular probe plus fixing vector pair to the stationary support for the purpose of making a micro-array is the result of magnetic interaction between the fixing vector and the support. It is important to observe that in this approach, the members of the pair constituted by the fixing support and the fixing vector do not both need to provide a magnetic force (or a magnetic field) simultaneously. It suffices for one of them to supply the force (or magnetic field) while the other one is capable of responding to said force (or of being attracted by the magnetic field).

Thus, the invention provides firstly an organized array of biological or chemical probes bonded to a support by magnetic coupling by means of a fixing vector. The support is preferably optically neutral.

In a particular embodiment of the invention, the support includes a permanent magnet, e.g. a samarium/cobalt or a neodymium/iron/boron magnet, such as those sold by the supplier Ugimag, 3830 Saint Pierre d'Allevard, France.

Alternatively, the support may present magnetization that is electrically induced.

In a particular embodiment of the invention, the support presents structured magnetization. If the support includes a permanent magnet, this property may be the result of the specific structure of the support. For example the support may be constituted by a slide of material that is magnetically inert (e.g. of silica) having holes that are regularly spaced apart and filled with ferromagnetic particles. The structured magnetization may also be obtained using a plane permanent magnet together with a mask. With an electromagnetic support, structured magnetization can be obtained, for example, by means of a microcircuit having a set of conductor loops in parallel.

The magnetic support may be selected in such a manner that the magnetic field is perpendicular or parallel to the major surface of the support.

The array of the present invention includes fixing vectors for coupling the biological and chemical probes to the magnetic support. By definition, these fixing vectors must be capable of responding to magnetic attraction or, where appropriate, of supplying it. A fixing vector is thus paramagnetic, superparamagnetic, or ferromagnetic. An example of a fixing vector is a bead of latex or of polysaccharides including particles of iron oxide.

The fixing vectors may be multipolar, or on the contrary they may be capable of taking up a particular orientation in a magnetic field. In a particular embodiment of the invention, the fixing vectors are themselves magnetized. Nevertheless, magnetized fixing vectors suffer from the drawback of clumping together even in the absence of an applied magnetic field. The fixing vectors are therefore preferably superparamagnetic.

The fixing vectors suitable for use in making arrays of the present invention have a diameter lying in the range 1 nanometer (nm) to 500 μm, and preferably lying in the range 0.5 μm to 5 μm.

The fixing vectors serve to provide coupling between the probes and the support. In addition to their magnetic properties, they must therefore be capable of bonding to the probes, by covalent bonding or by strong interactions, e.g. of the affinity type. In a preferred embodiment of the invention, the fixing vector carries elements capable of bonding specifically to a biological target. An example of a covalent bond specific to a particular type of target is the bond established by condensation between a Schiff base and certain compounds of the R—NH₂ type. Examples of non-covalent bonds that are specific to a biological target are affinity interactions established between a receptor and a corresponding ligand, or between an antigen and an antibody that recognizes it. The bond may be also be constituted by hydrogen bonds established between two complementary nucleic acid sequences. Below in this text, a fixing vector is said to carry elements capable of bonding specifically to a biological target when it carries elements having high affinity for a given type of biological molecule. Depending on the type of probe in question, such elements may be selected, for example, from the group comprising by immunoglobulins, antigens or fragments thereof, membrane receptors, membrane receptor ligands, avidin, streptavidin, poly-T or poly-U oligonucleotides, or any chemical or biological molecule that makes specific interaction possible.

The invention also provides the product of coupling between a biological or chemical probe and a fixing vector. Depending on the nature of the probe, the number of examples of the probe coupled to each fixing vector may lie in the range 1 (e.g. when the probe is a cell) to 108 (e.g. for a nucleic probe). In particular, the invention provides a pair produced by coupling a nucleic acid probe and a fixing vector. The nucleic acid probe is preferably a single-strand DNA probe of length greater than 50 oligonucleotides, but it may also be a single-strand oligonucleotide or double-strand DNA. Each pair produced by coupling a fixing vector and a nucleic acid probe carries a number of nucleic probes lying in the range 10³ to 10⁸, and preferably in the range 10⁵ to 10⁷.

The use of a pair produced by coupling a biological or chemical probe and a fixing vector for making an organized array of biological or chemical probes, also forms an integral part of the invention.

In arrays of the invention, each probe coupled to its fixing vector is deposited at a spot of diameter lying in the range 10 μm to 1 mm, and preferably in the range 50 μm to 200 μm. Spot diameter is selected by the person skilled in the art as a function of the intended application, in particular as a function of the type of probe used.

Each spot of an array of the invention carries 10⁵ to 10¹⁰ probes, and preferably 10⁸ to 10¹⁰ probes. The number of probes per spot is the product of the number of fixing vectors per spot multiplied by the average number of probes bonded to a fixing vector. These two parameters can be verified by experiment. The average number of probes bonded to a fixing vector depends in particular on the “capacity” of the vector, i.e. the average number of bonding sites on each vector. This “capacity” is specified by the suppliers of functionalized magnetic beads. The number of fixing vectors per spot is calculated by the person skilled in the art so that the density of fixing vectors per unit area is such that the fixing vectors form a monolayer. The arrays of the invention can thus advantageously have spots of density that is uniform, i.e. each spot ideally has the same number of probes.

The density of arrays of the invention lies in the range 1 to 100,000 spots per cm², and preferably in the range 10 to 1000 spots per cm².

The invention also provides methods of manufacturing an organized array of biological or chemical probes bonded to a support by magnetic coupling by means of a fixing vector. A first method of the invention, described in Example 1, comprises the following steps:

A) bonding probes to the fixing vector; and

B) depositing probes coupled to the fixing vector on a magnetic support in an organized array.

Another method of manufacture, illustrated in Example 2, of an organized array of biological or chemical probes bonded to a support by magnetic coupling by means of a fixing vector comprises the following steps:

A) magnetically coupling a fixing vector at known coordinates on a support; and

B) bonding probes to the fixing vectors.

In the above-described method, the step of coupling the fixing vector to the support can be performed by using a mask.

This mask is pierced by windows that are regularly spaced apart and is preferably opaque to the magnetic field such that the fixing vectors become fixed solely in said windows. Alternatively, the mask may be permeable to the magnetic field. Under such circumstances, the fixing vectors will initially become fixed over the entire surface of the support regardless of whether it is covered by the mask. Subsequently, the mask is carefully removed so as to remove the fixing vectors that are not directly in contact with the support.

An alternative method of manufacturing an organized array of biological or chemical probes bonded to a support by magnetic coupling by means of a fixing vector is described in Example 3, and comprises the following steps:

A) bonding probes to the fixing vector;

B) depositing probes coupled to a fixing vector on a non-magnetized slide, in an organized array; and

C) transferring the array of step B) onto a magnetic support.

In all of the methods of the invention as described above, the fixing vectors, regardless of whether they are coupled to the probes, are deposited on the optionally magnetic support using any means that can be devised by the person skilled in the art. For example this may be done using pipettes, micropipettes, piezoelectric pipettes, nozzles, needles, or small electromagnets having soft iron cores, each constituted by a needle of diameter smaller than 200 μm at its tip.

A particular method of the invention, described in Example 4, serves to manufacture an organized array of probes constituted by single-strand nucleic acid molecules of size that may lie in the range 20 to 5000 nucleotides, and preferably in the range 100 to 500 nucleotides. This method comprises the following steps:

A) amplifying probes from nucleotide primers, at least one of which is biotinylated;

B) denaturing the probes by heating, followed by fast cooling;

C) coupling the biotinylated probes on beads that are paramagnetic, superparamagnetic, or ferromagnetic, and bonded to avidin or streptavidin;

D) magnetically precipitating the probes bonded to the beads;

E) removing unbonded probes by washing;

F) resuspending the probes at a selected concentration; and

G) depositing the probes on a support.

In a preferred implementation of the above-described methods, the step of bonding the probe to the fixing vector is performed at a saturating concentration of probes for the fixing vector. This makes it possible in particular to know the average number of probes bonded to each fixing vector, and to do so in a manner that is easily reproducible.

The invention also provides a support capable of presenting structured magnetization that is permanent or electrically induced, for the purpose of manufacturing an array of biological or chemical probes of the kind described above. A preferred support of the invention presents structured magnetization that is permanent. In a manner that is even more preferred, a support of the invention presents static magnetization that is decoupled from any electric field and that is inert for any charged molecule. Most biological polymers, and in particular DNA, are charged and are therefore sensitive to electric field, even at very low voltage, as described in the articles by Meller et al. (A. Meller, L. Nivon, et al. (2000), “Rapid nanopore discrimination between single polynucleotide molecules” Proc. Natl. Acad. Sci. USA, 97(3): 1079-84; and A. Meller, L. Nivon, et al. (2001), “Voltage-driven DNA translocations through a nanopore”, Phys. Rev. Lett., 86(15): 3435-8). A magnetic field induced by means of a coil is generally accompanied by a residual electric field (depending on the shape of the coil). The use of an electromagnetic field induced by a coil of a shape that does not enable said residual magnetic field to be canceled out completely therefore results in non-specific adsorption of charged molecules on the surface of the support. In addition, such an electric field might modify the physico-chemical characteristics of the molecules. For example, under certain conditions, the ion gradient induced by the electric field might deteriorate the DNA helices.

An organized array of fixing vectors coupled to a support by magnetic interaction between the support and the fixing vectors also forms part of the present invention.

Finally, the invention provides a kit for making an organized array of biological or chemical probes, the kit comprising a magnetic support and fixing vectors. Such a kit is particularly advantageous for enabling research or analysis laboratories to make their own arrays of probes that are particularly adapted to their own requirements, and to do so at low cost. For example, the kit may enable a laboratory to produce a “made-to-measure” DNA chip, possibly by using the last of the methods mentioned above.

In preferred but non-limiting manner, the magnetic support present in a kit of the invention is optically neutral and includes a permanent magnet. Where appropriate, the support may present structured magnetization.

In a particular version of a kit of the invention, the fixing vectors are already fixed to the support in an organized array.

The fixing vectors present in a kit of the invention are constituted, for example, by paramagnetic, superparamagnetic, or ferromagnetic beads.

In an advantageous embodiment of a kit of the invention, the fixing vector is functionalized, i.e. it carries elements capable of bonding specifically with a biological target.

Kits of the invention may be designed for several types of use, or they may be designed for use with a given type of probe. For example, a kit having as its fixing vectors beads carrying oligonucleotides with a poly-T or poly-U at one end is more specifically for use in making messenger RNA or cDNA chips onto which a poly-A tail is grafted. In contrast, a kit in which the fixing vector is coupled to streptavidin or to avidin is suitable for use with any type of probe that can be biotinilated.

Kits of the invention may also have one or more control probes, optionally bonded to the fixing vector. For example this may be constituted by a single-strand DNA probe encoding a fragment of β-actin, if the kit is intended for making chips for analyzing the expression of genes in eukarytic cells. A kit may also include both the control probe in the free state and the control probe already fixed to the fixing vector, thus enabling the user to monitor the step of coupling probes to the fixing vectors. Kits may also include fixing vectors coupled to marked probes for calibrating deposition of fixing vectors on the support. These examples are not limiting and the person skilled in the art is capable of devising any type of kit having additional elements for making it easier to use, for enabling results to be interpreted more precisely, or for targeting a particular type of application.

The following examples and figures show how the present invention can be implemented and the advantages thereof, but without limiting its scope.

Legends for the figures:

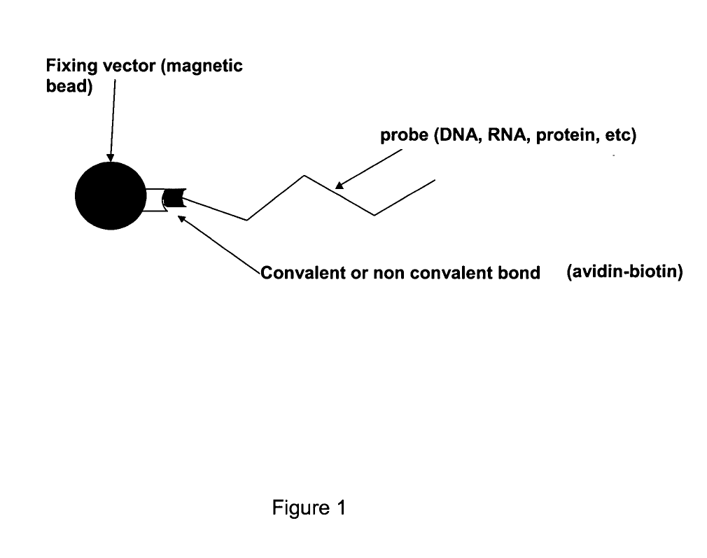

FIG. 1: fixing the probe to the magnetized particle.

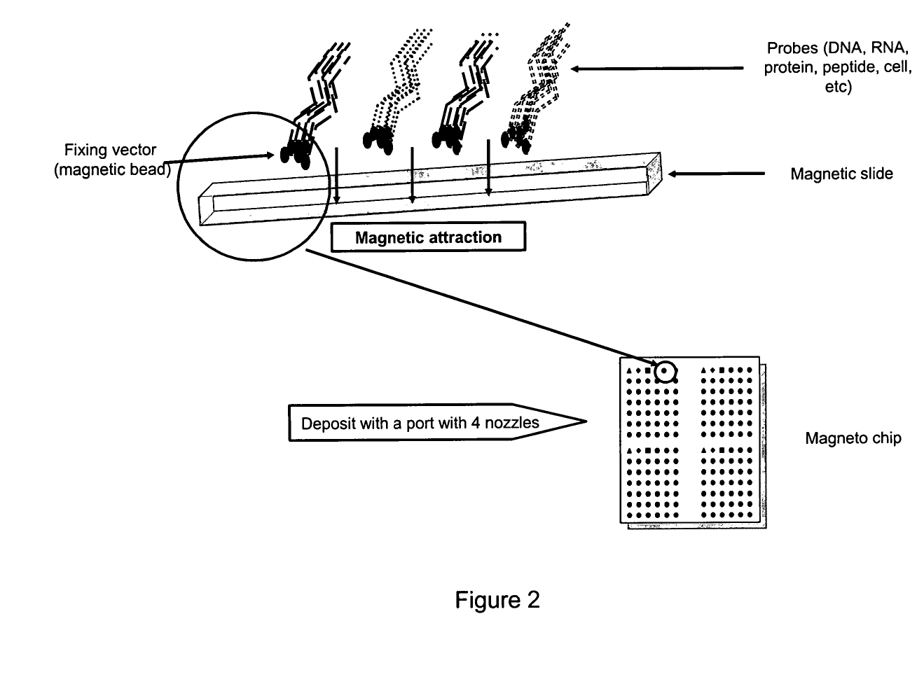

FIG. 2: making a magnetic chip.

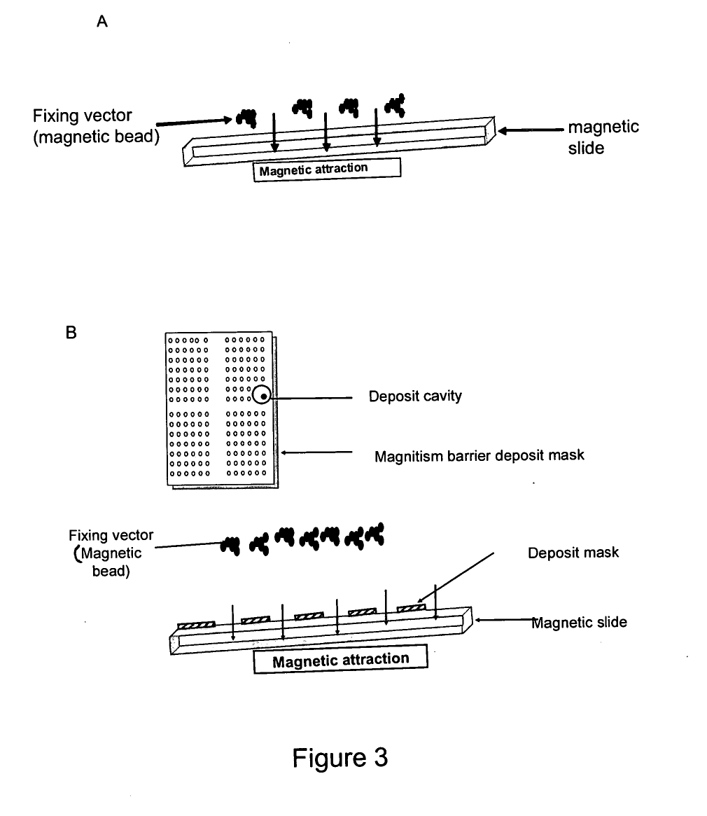

FIG. 3: making an array of functionalized beads:

A) by successive deposits;

B) by deposition in a single stage using a mask that is impermeable to the magnetic field.

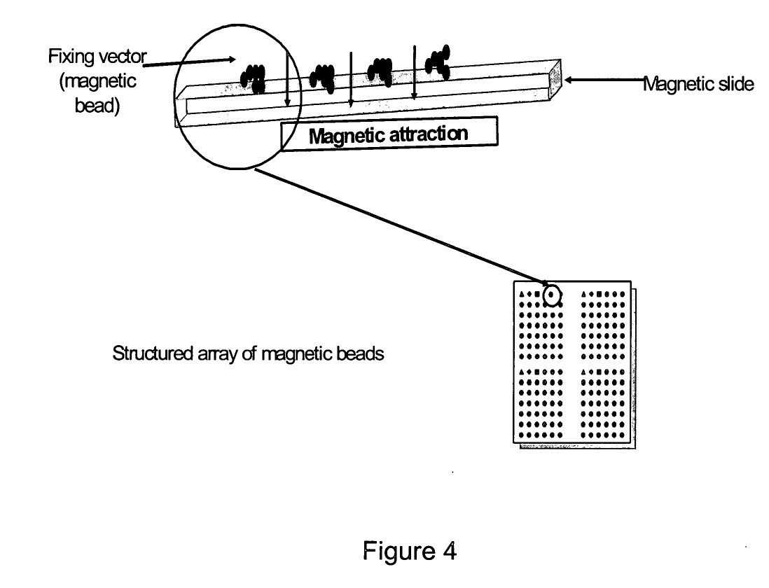

FIG. 4: organized array of magnetic beads.

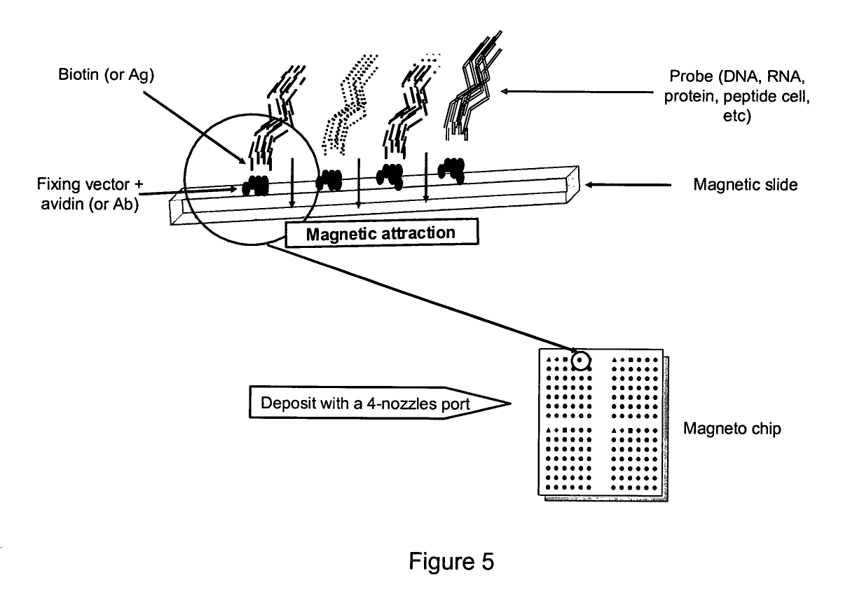

FIG. 5: depositing probes onto an array that has previously been magnetically organized.

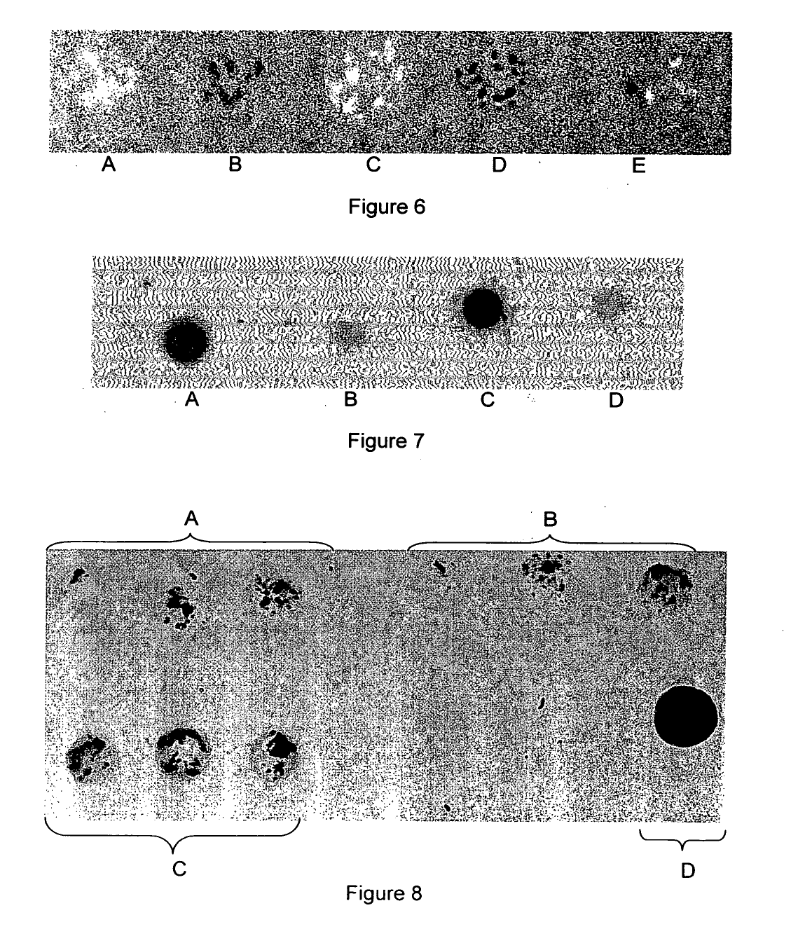

FIG. 6: fluorescent image scanned at a resolution of 5 μm showing the formation of a complex between nucleic probes and fixing vectors on the slide:

A) Cy3-marked probe, not functionalized with biotin;

B) Cy3-marked probe, functionalized with biotin at 5′;

C) Cy3-marked probe, not functionalized;

D) Cy3-marked probe, functionalized with biotin at 5′;

E) single particle. Observation was performed using a GMS 428 scanner at a resolution of 5 μm.

FIG. 7: fluorescent image scanned at a resolution of 5 μm showing hybridization of the nucleic target on the bead-and-probe complex:

A) unmarked probe functionalized with biotin at 5′, target marked with Cy3;

B) probe not functionalized with biotin, target marked with Cy3;

C) unmarked probe functionalized with biotin at 5′, target marked with Cy5;

D) probe not functionalized with biotin, target marked with Cy5. Observation was performed using a GMS 428 scanner at resolution of 5 μm.

FIG. 8: fluorescent image scanned at a resolution of 5 μm showing the fixing of a protein probe and target on a fixing vector:

A) particles preincubated with bovine serum albumin (BSA) and then with total protein total extract marked with Cy3;

B) particles preincubated with non-specific antibody and then with non-marked BSA, and then with Cy3-marked total extract;

C) particles preincubated with specific antibody x, then with unmarked BSA, then with Cy3-marked total extract;

D) Cy3 marked protein deposited after washing. Observation was performed using a GMS 428 scanner at resolution of 5 μm.

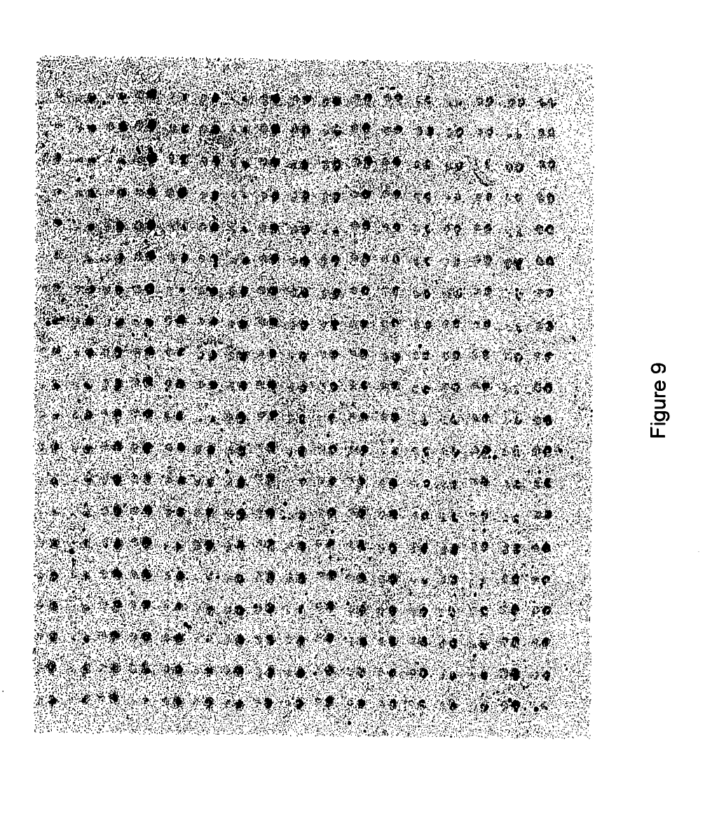

FIG. 9: an array of beads functionalized with Cy3-marked probes at a density of 625 per cm², observed using a GMS 428 scanner at a resolution of 5 Am. The scanned surface was a square having a side of 0.75 cm.

### EXAMPLE 1

### Method of Making a Magnetic Chip by Single-Step Deposition

The probes were fixed to magnetized particles, e.g. multipolar magnetic beads. Fixing to the bead was performed either by a covalent bond, or by any other chemical bond, e.g. by establishing a Schiff base, or by a non-covalent bond, such as, for example, streptavadin/biotin or antibody/antigen bonds (FIG. 1).

The pair comprising the fixing vector and the probes was then deposited at the desired density on a small area of a few μm² of the magnetized support. This provided, at determined coordinates on the support, a spot of identical probes at the desired density. The operation was performed as often as necessary to obtain a micro-array of spots of different probes. It should be observed that a plurality of spots can be made simultaneously (FIG. 2).

### EXAMPLE 2

### A Method of Making a Magnetic Chip by Deposition in Two Steps

An alternative to the protocol described in Example 1 is to use a non-covalent bond between the fixing vectors and the probes. Under such circumstances, it is possible to separate depositing functionalized beads and probes on the magnetic surface. Thus, by way of example, beads functionalized with streptavidin were initially deposited on the magnetic support so as to create a regular array of magnetic beads in which each spot was constituted by beads at a well-defined density. The array was made in two different ways, either by successive depositions as above (FIG. 3A), or else in a single operation using a mask (FIG. 3B).

The array of magnetic beads was made using a mask that isolates magnetic fields, leaving unmasked only those areas of the magnetized slide that were to receive the beads, the remainder of the surface of the magnetized slide remaining masked (cf. FIG. 3B). As a result, only the unmarked portions of the magnet could fix magnetic beads. The mask for depositing beads can be constituted by a silica slide structured by chemical treatment or by a plastics film machined by means of a laser.

For a support presenting structured magnetization, e.g. a silica slide having micro-magnets embedded therein, the array of fixing vectors can be made in a single operation without it being necessary to use a mask.

Thus, it is possible in a single deposition operation to obtain an array of organized magnetic beads (FIG. 4).

Thereafter, in a second step, specific probes coupled to biotin at a saturating concentration for streptavadin were deposited on each spot of magnetic beads (FIG. 5). The same strategy can be used for all non-covalent bonds between fixing vectors and probes.

After deposition, non-fixed probes were eliminated by adding free streptavidin and washing.

This produced a micro-array of spots, themselves constituted by specific probes at the desired density durably fixed to the support at determined coordinates.

### EXAMPLE 3

### Method of Making a Magnetic Chip by Replication

A final variant consists in depositing probes bonded to fixing vectors onto a glass slide and then in transferring the pairs constituted by the magnetic beads and the probes fixed thereto to a magnetized slide.

Transfer can be performed merely by moving the magnetized slide over the glass slide, with pairs constituted by magnetized beads and probes jumping spontaneously to equivalent coordinates from the glass slide to the magnetized slide.

### EXAMPLE 4

### Preparing and Using a Magnetic Chip of Single-Strand DNA Probes

Preparing the Slides

In an initial manufacturing step, magnetic attraction was provided by magnetized slides having dimensions of 35 mm×25 mm and a thickness of 1 mm.

Two types of composition for the slides were tested, firstly slides made of neodymium iron boron capable of providing maximum magnetization of 1.3 teslas (T), and secondly samarium cobalt magnetized slides capable of supplying maximum magnetization of 1 T.

For each type of slide, two orientations for the magnetic field were tested: firstly a field perpendicular to the major surface of the slide, and secondly magnetization parallel to the major surface of the slide. For each field, four different forces were used: 0.2 T, 0.5 T, and either 1 T or 1.3 T.

Fixing vector

In a first experiment, the fixing vectors used were beads (Dynabeads) having a diameter of 2.8 μm, made of a polymer including iron oxides (Fe₃O₃ at 10%-14%) with susceptibility of 8×10⁻³ cgs per unit. The oxide particles provided the ability to react to the magnetic field. The beads were covered in covalent manner with streptavidin molecules capable of fixing biotin. The average density of the streptavidin receptors was 7×10⁵ to 10⁶ per bead.

Depositing Spots or Hybridization Units

Pairs comprising vector (bead) and probe (single-strand cDNA molecule) were deposited initially using a pen with a 0.2 mm tip. The concentration of beads selected for each deposit was such that a deposit contained about 6000 beads, corresponding to 6×10⁹ probe molecules per hybridization unit, assuming that all of the streptavidin sites were saturated. Under such conditions, a monolayer deposit of beads was obtained (each bead resting directly on the slide).

Preparing Probes

Starting from pairs of primers biotinylated at 5′, either on the forward primer or on the reverse primer or both primers, amplification was performed by polymerized chain reaction (PCR) of the specific region of the gene under study. Thus, for each PCR, a double-stranded sequence was obtained. The sequence was biotinylated respectively at 5′ of the coding strand, and at 5′ of the reverse standard, or was biotinylated at 5′ on both strands, depending on the selected construction. In both cases, the double strands were denatured by soaking, and then coupled to magnetic beads functionalized with streptavidin. The number of magnetic beads added was determined in such a manner that the streptavidin sites were saturated by the biotin of the DNA. As a result, all of the beads fixed n single-strand DNA molecules, where n represents the number of streptavidin sites per bead. Bead and single-strand DNA pairs were precipitated in a magnetic field and then washed to remove any DNA that was not fixed to biotin (when only one of the primers was biotinylated, only one of the two strands of double-strand DNA is retained). Pairs comprising magnetic beads (vectors) and single-strand DNA (probes) were resuspended at the desired concentration in order to obtain a final desired concentration of DNA; under such circumstances, the molar concentration of DNA is obtained directly since it depends on the concentration of beads.

Preparing the Array

The pair comprising the fixing vector (magnetic bead) and the probes (single-strand DNA) was deposited by means of a pen or a piezoelectric pipette at the desired density on a small area having a side of 100 μm to 300 μm. This ensures that hybridization units of identical probes at the desired density were obtained at determined coordinates on the support. The operation was performed as often as necessary to obtain a micro-array of spots of different probes. It should be observed that a plurality of spots can be made simultaneously.

Hybridizing

The slide made in this way was then hybridized directly with a mixture of fluorescence-marked cDNA. The cDNA was obtained by RT-PCR (in the presence of Cy3™ for example, as the fluorescent marker), from the mRNA extracted from the cells under study. It would also be possible to perform co-hybridization using two extracts of RNA supplying cDNA marked respectively with Cy3™ and Cy5™.

After the slide had been washed to eliminate non-hybridized cDNA molecules, the quantity of fluorescence at each spot indicated the amount of hybridization, and thus the proportion of each type of molecule.

This marking is not limiting; the same experiments could be performed using radioactive cDNA.

### EXAMPLE 5

### Magnetic Chip of Single-Strand DNA Probes for Screening GAPDH and HPRT Genes

A magnetic chip was made with probes using the following sequences as obtained by specific amplification (PCR). For each sequence, the three structures described above were made:

sequence 5′ biotinylized on the coding strand;

sequence 5′ biotinylized on the antisense strand;

sequence 5′ biotinylized on the both strands.

The sequences that are underlined correspond to the specific primers used to amplify the probes.

Probes Selected for the GAPDH Gene

CTGGTGTCTTCACCACCATGGAGAAGGCCGGGGCCCACTTGAAGGGTG
GAGCCAAACGGGTCATCATCTCCGCCCCTTCTGCCGATGCCCCCATGT
TTGTGATGGGTGTGAACCACGAGAAATATGACAACTCACTCAAGATTGT
CAGCAATGCATCCTGCACCACCAACTGCTTAGCCCCCCTGGCCAAGGT
CATCCATGACAACTTTGGCATTGTGGAAGGGCTCATGACCACAGTCCAT
GCCATCACTGCCACCCAGAAGACTGTGGATGGCCCCTCTGGAAAGCTG
TGGCGTGATGGCCGTGGGGCTGCCCAGAACATCATCCCTGCATCCACT
GGTGCTGCCAAGGCTGTGGGCAAGGTCATCCCAGAGCTGAACGGGAA
GCTCACTGGCATGGCCTTCCGTGTTCCTACCCCCAATGTGTCCGTCGTG
GATCTGACGTGCCGCCTGGAGAAACCTGCCAAGTATGATGACATCAAG
AAGGTGGTGA
TTCACCACCATGGAGAAGGCCGGGGCCCACTTGAAGGGTGGAGCCAAA
CGGGTCATCATCTCCGCCCCTTCTGCCGATGCCCCCATGTTTGTGATGG
GTGTGAACCACGAGAAATATGACAACTCACTCAAGATTGTCAGCAATGC
ATCCTGCACCACCAACTGCTTAGCCCCCCTGGCCAAGGTCATCCATGAC
AACTTTGGCATTGTGGAAGGGCTCATGACCACAGTCCATGCC

Probes Selected for the HPRT Gene

GCTGGTGAAAAGGACCTCTCGAAGTGTTGGATACAGGCCAGACTTTGTT
GGATTTGAAATTCCAGACAAGTTTGTTGTTGGATATGCCCTTGACTATAA
TGAGTACTTCAGGAATTTGAATCACGTTTGTGTCATTAGTGAAACTGGAA
AAGCCAAATACAAAGCCTAAGATGAGCGCAAGTTGAATCTGCAAATACG
AGGAGTCCTGTTGATGTTGCCAGTAAAATTAGCAGGTGTTCTAGTCCTG
TG
AGGAGATGGGAGGCCATCACATTGTGGCCCTCTGTGTGCTCAAGGGGG
GCTATAAGTTCTTTGCTGACCTGCTGGATTACATTAAAGCACTGAATAGA
AATAGTGATAGATCCATTCCTATGACTGTAGATTTTATCAGACTGAAGAG
CTACTGTAATGATCAGTCAACGGGGGACATAAAAGTTATTGGTGGAGAT
GATCTCTCAACTTTAACTGGAAAGAATGTCTTGATTGTTGAAGATATAAT
TGACACTGGTAAAACAATGCAAACTTTGCTTTCCCTGGTTAAGCAGTACA
GCCCCAAAATGGTTAAGGTTGCAAGCTTGCTGGTGAAAAGGACCTCTCG
AAGTGTTGGATACAGGCCAGACTTTGTTGGATTTGAAATTCCAGACAAG
TTTGTTGTTGGATATGCCCTTGACTATAATGAGTACTTCAGGAATTTGAA
TCACGTTTGTGTCATTAGTGAAACTGGAAAAGCCAAATACAAAGCC

### EXAMPLE 6

### Verifying the Formation of a Complex between the Nucleic Probes and the Fixing Vectors on the Slide

The fixing vectors used herein were paramagnetic Fe₂O₃ polystyrene particles having a diameter of 1 μm, functionalized with streptavidin. The probe corresponded to the fragment of the GAPDH gene given in the preceding example. It was functionalized or not functionalized by biotin at 5′ on only one of its two strands (the sense strands).

Primers defining the sequence:

5′CTGGTGTCTTCACCACCATG3′
5′TCACCACCTTCTTGATGTCATC3′

The probes were marked either with Cy3™ or with Cy5™ all along the sequence during amplification by PCR.

The products of the PCR were precipitated, resuspended, denatured at 100° C., soaked at 0° C., and then soaked with the particles at 4° C. for 60 minutes. The resulting probe-and-vector complexes were precipitated by magnetic activation and they were washed three times in 10×SSC buffer and once in 3×SSC buffer. The residue was resuspended in a 3×SSC buffer.

An aliquot of the resulting solution was taken using a needle and deposited on a 1 T magnetic slide having dimensions 20 mm×30 mm×1 mm. The deposit was rinsed three times in 10×SSC buffer.

FIG. 6 shows the results of the following depositions:

A) Cy3-marked probe not functionalized with biotin;

B) Cy3-marked probe functionalized with biotin at

C) Cy3-marked probe, not functionalized;

D) Cy3-marked probe functionalized with biotin at

E) single particle.

This figure shows that the fixing of probes on a particle is indeed specific (no fluorescent marking when the probe is not functionalized with biotin), and that the bead-and-probe complex is indeed retained on the magnetic slide (Figures B and D).

### Example VII

### Verifying Hybridization of the Nucleic Target on the Bead-and-Probe Complex

The probes were constituted by a fragment of the sequence for the GAPDH gene functionalized or not functionalized by biotin at 5′ on the sense strand.

The targets were constituted by the PCR product of GAPDH marked all along the sequence with Cy3™ or Cy5™.

The fixing vectors were paramagnetic Fe₂O₃ polystyrene particles having a diameter of 1 μm and functionalized by streptavadin.

The probes were denatured at 100° C., soaked at 0° C., then complexed with the particles at 4° C. for 60 min. The resulting probe-and-vector complexes were precipitated by magnetic activation and rinsed four times in 10×SSC buffer. The residue was resuspended in a 25% formamide buffer, the denatured targets were then mixed with the probes and they were hybridized at 50° C.

An aliquot of the resulting solution was taken using a needle and deposited on a 1 T magnetic slide having dimensions of 20 mm×30 mm×1 mm. The deposit was rinsed three times using a 10×SSC buffer and once with a 3×SSC buffer.

The results are shown in FIG. 7, where the various spots correspond to the following conditions:

A) unmarked probe functionalized with biotin at 5′, target marked with Cy3;

B) probe not functionalized with biotin, target marked with Cy3;

C) unmarked probe functionalized with biotin at 5′, target marked with Cy5;

D) probe not functionalized with biotin, target marked with Cy5.

The targets were thus capable of hybridizing on the probe when fixed to the particle, and there was very little non-specific absorption.

The target was indeed retained on the slide by means of the vector.

### Example VIII

### Verification that the Target and the Protein Probe are fixed to the Fixing Vector

The vectors were paramagnetic Fe₂O₃ polystyrene particles having a diameter of 1 μm, capable of asorbing proteins on their surface in non-specific manner.

The probe in this case was an antibody specific to a protein x.

The target was a protein x contained in a cellular extract marked with Cy3.

A non-specific antibody of the protein x that does not recognize any element of the cellular extract containing the target was used as a reference.

Various different hybridizations were performed, and the results appear in FIG. 8:

FIG. 8A: the particles were preincubated with serum albumin (BSA), and then with the Cy3-marked total protein extract.

Non-specific fixing can be seen.

FIG. 8B: the particles were preincubated with the non-specific antibody, and then saturated with the non-marked BSA, and then incubated with the Cy3-marked total extract.

The same non-specific fixing can be seen as appears during pre-hybridization with BSA.

FIG. 8C: the particles were preincubated with the antibody specific to x, saturated with non-marked BSA, and then incubated with the Cy3-marked total extract.

The intensity of the fluorescent signal was greater than that of non-specific hybridization. Specific hybridization had indeed taken place with the anti-x antibody.

The non-specific fixing observed in this experiment was large. This non-specific fixing can be eliminated by using paramagnetic particles of the silicate or silicon type such as “silica particul” or “beads silica” having SH functions grafted thereon.

Those particles do not adsorb proteins, and they thus eliminate non-specific adsorption. The antibodies can be fixed, for example, by the thiol of the heavy chain after reduction. The specific fixing site can then be constituted by a light chain and a heavy chain bonded covalently to the particle by an S-S bridge.

### Example IX

### Feasibility of a Paramagnetic Particle Array on a Support Possessing Static Magnetization at 1 T

Beads functionalized with Cy3-marked probes were deposited at a density of 625 per cm² on a support presenting static magnetization at 1 T. The resulting array was then washed three times with a 10×SSC solution, then once with a 3×SSC solution. The result is shown in FIG. 9, showing that a stable array was obtained.

1/ An organized array of biological or chemical probes bound to a support by magnetic coupling using a fixing vector.

2/ An array according to claim 1, fixed to a support that is optically neutral.

3/ An array according to claim 1 or claim 2, fixed to a support that includes a permanent magnet.

4/ An array according to claim 1 or claim 2, fixed to a support that presents electrically-induced magnetism.

5/ An array according to any one of claims 1 to 4, fixed to a support presenting structured magnetism.

6/ An array according to any one of claims 1 to 5, in which the magnetic field is perpendicular or parallel to the plane of the support.

7/ An array according to any one of claims 1 to 6, in which the fixing vector is paramagnetic, superparamagnetic, or ferromagnetic.

8/ An array according to claim 7, in which the fixing vector is a bead of latex or of polysaccharides including particles of iron oxide.

9/ An array according to claim 7 or claim 8, in which the fixing vector is a multipolar bead.

10/ An array according to claim 7 or claim 8, in which the fixing vector is capable of orienting itself in a magnetic field.

11/ An array according to claim 10, in which the fixing vector is itself magnetized.

12/ An array according to any one of claims 7 to 11, in which the fixing vector has a diameter lying in the range 1 nm to 500 μm, and preferably in the range 0.5 μm to 5 μm.

13/ An array according to any one of claims 1 to 12, in which the fixing vector carries elements capable of bonding specifically to a biological target.

14/ An array according to claim 13, in which the elements capable of bonding specifically to a biological target carried by the fixing vector are selected from the group comprising: immunoglobulins, antigens, membrane receptors, membrane receptor ligands, avidin, streptavidin, poly-T or poly-U oligonucleotides.

15/ An array according to any one of claims 1 to 14, in which each probe is deposited as a spot of diameter lying in the range 10 μm to 1 mm, and preferably in the range 50 μm to 200 μm.

16/ An array according to any one of claims 1 to 15, in which each spot has 10⁵ to 10¹⁰ probes, and preferably 10⁸ to 10¹⁰ probes.

17/ An array according to any one of claims 1 to 16, having a density of 1 to 100,000 spots per cm², and preferably of 10 to 1000 spots per cm².

18/ A method of manufacturing an organized array of biological or chemical probes bonded to a support by magnetic coupling by means of a fixing vector, the method comprising the following steps:
A) bonding probes to the fixing vector; and
B) depositing probes coupled to the fixing vector on a magnetic support in an organized array.

19/ A method of manufacturing an organized array of biological or chemical probes bonded to a support by magnetic coupling by means of a fixing vector, the method comprising the following steps:
A) magnetically coupling a fixing vector at known coordinates on a support; and
B) bonding probes to the fixing vectors.

20/ A method according to claim 10, characterized in that the step of coupling the fixing vector to the support is performed by using a mask.

21/ A method of manufacturing an organized array of biological or chemical probes bonded to a support by magnetic coupling by means of a fixing vector, the method comprising the following steps:
A) bonding probes to the fixing vector;
B) depositing probes coupled to a fixing vector on a non-magnetized slide, in an organized array; and
C) transferring the array of step B) onto a magnetic support.

22/ A method according to claim 18 or claim 21, for making an organized array of probes constituted by single-strand nucleic acid molecules, the method comprising the following steps:
A) amplifying probes from nucleotide primers, wherein at least one of which is biotinylated;
B) denaturing the probes by heating, followed by fast cooling;
C) coupling the biotinylated probes on beads that are paramagnetic, superparamagnetic, or ferromagnetic, and bonded to avidin or streptavidin;
D) magnetically precipitating the probes bonded to the beads;
E) removing unbonded probes by washing;
F) resuspending the probes at a selected concentration; and
G) depositing the probes on a support.

23/ A method according to any one of claims 18 to 22, in which the step of bonding the probe to the fixing vector is performed with a concentration of probes that is saturating for the fixing vector.

24/ A support presenting structured permanent magnetization for making an array of biological or chemical probes, using a method according to any one of claims 18 to 23.

25/ A support according to claim 24, in which the structured magnetization is the result of static magnetization decoupled from any electric field and is inert for any charged molecule.

26/ An organized array of fixing vectors coupled to a support by magnetic interaction between the support and the fixing vectors.

27/ A pair produced by coupling a nucleic acid probe and a fixing vector, in which a fixing vector is coupled to a number of nucleic acid molecules lying in the range 10³ to 10⁸, and preferably in the range 10⁵ to 10⁷.

28/ The use of a pair produced by coupling a biological or chemical probe and a fixing vector, to make an organized array of biological or chemical probes according to any one of claims 1 to 17.

29/ A kit for making an organized array of biological or chemical probes, the kit comprising a magnetic support and fixing vectors.

30/ A kit according to claim 29, in which the fixing vector carries elements capable of bonding specifically to a biological probe.
